(19) United States
(12) Patent Application Publication
(10) Pub. No.: US 2018/0110847 A1
(43) Pub. Date: Apr. 26, 2018

(54) Immunotherapy of Cancer by Induction of Immunologically-Mediated Selective Killing of Tumor Vasculature Using Modified Endothelial Cells and Progenitors Thereof

(71) Applicant: BATU BIOLOGICS, INC., San Diego, CA (US)

(72) Inventors: Samuel C. Wagner, San Diego, CA (US); Thomas E. Ichim, San Diego, CA (US)

(21) Appl. No.: 15/789,260

(22) Filed: Oct. 20, 2017

Related U.S. Application Data
(60) Provisional application No. 62/410,507, filed on Oct. 20, 2016.

(51) Int. Cl. A61K 39/00; C12N 5/073

(52) U.S. Cl. CPC A61K 39/0011; C12N 5/0605; C12N 2500/40; C12N 2501/24; C12N 2500/00

(57) ABSTRACT

Disclosed are methods, protocols, and compositions of matter useful for treatment of cancer by elicitation of immunity towards tumor blood vessels. In one embodiment, the invention teaches the stimulation of expression of tumor blood vessel associated antigens in cells derived from endothelial progenitor cells, through culture of cells in conditions resembling the tumor microenvironment. In another embodiment, the invention teaches increasing immunogenicity of endothelial progenitor cells or progeny thereof through treatment with agents such as interferon gamma, which are capable of upregulating histocompatibility antigens, which allow for allogeneic rejection upon administration. In another embodiment, the invention teaches immunization with endothelial progenitor cells or progeny thereof treated under conditions to resemble tumor blood vessels, in which administered cells are purposely mismatched with recipient HLA in order to provide for enhanced allogenicity. In one embodiment, purposeful mismatching is accomplished through transfection or “cell painting” of molecules capable of eliciting an immunological response.

### BACKGROUND

The role of the immune system in controlling cancer has been suggested by studies which demonstrate higher incidence of cancer in patients that are chronically immune suppressed. The notion that the immune system plays a role in controlling oncogenesis was described in the 1960s by Burnet, who noticed higher levels of malignancies in patients that suffer from inborn immunodeficiences. This has subsequently been verified over the decades in a variety of neoplasias, and in a variety of immune deficiencies. An example of the impact that congenital immunodeficiency has on incidence of cancer can be seen in a study of 377 patients with primary hypogammaglobulinaemia, mainly common variable immunodeficiency (CVID), 316 patients survived the first 2 years after diagnosis and were the subject of a study of cancer incidence. Among the 220 patients with CVID, there was a 5-fold increase of cancer due mainly to large excesses of stomach cancer (47-fold) and lymphomas (30-fold). The excess of stomach cancer is probably related to the high frequency of achlorhydria in CVID. 3 of the 7 patients with stomach cancer and CVID survived for 5 years or longer. In another study, a 30-fold increase in incidence of colorectal cancer was observed in patients suffering from X-linked agammaglobulinaemia (XLA). Interestingly, in patients with CVID, the patients that have lower CD8 T cell numbers and activity are more susceptible to cancer development.

Immune recognition of cancers is further supported by studies in which infiltration of tumors by lymphocytes is associated with enhanced survival. This has been demonstrated in many tumor types, for example in breast, gallbladder, and ovarian.

The role of cancer immunotherapy is to augment the existing process of tumor infiltrating lymphocytes so as to ensure regression of tumor. The first part of the tumor that lymphocytes encounter during an immunological attack is the tumor endothelium. The tumor endothelium is known to be antigenically unique based on expression of molecules not found on healthy endothelium. Known markers include TEM-1 (endosialin), and CD1.

### SUMMARY OF THE INVENTION

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented hereinbelow. It is to be understood that both the following general description and the following detailed description are exemplary and explanatory only and are not restrictive.

The invention provides means of utilizing endothelial progenitor cells and products derived from the endothelial progenitor cells as a cancer vaccine which selectively induces immunity towards tumor vasculature and not healthy, non-malignant, vasculature. In accordance with the embodiments disclosed herein, the method comprises obtaining endothelial progenitor cells and culturing the cells under conditions resembling the tumor microenvironment. The products of the cultured endothelial cells are administered in a manner to stimulate an immune response capable of cross-reacting with tumor associated endothelial cells.

In one embodiment, the endothelial progenitor cells are derived from placental tissue. In another embodiment, endothelial progenitor cells are HLA mismatched to the cancer patient in need of treatment.

In yet another embodiment, the endothelial progenitor cells are treated with interferon gamma at concentrations and duration sufficient to increase expression of HLA I and HLA II. In one embodiment, the endothelial progenitor cells or progeny thereof are rendered metabolically similar to tumor endothelial cells by culture under hypoxic conditions.

In still another embodiment, the endothelial progenitor cells or products thereof are generated to resemble tumor endothelial cells by culture in acidic conditions. In one embodiment, the acidic conditions comprise a pH of less than about 7. In another embodiment, the acidic conditions comprise a pH of about 5 to about 7. In one embodiment, the acidic conditions comprise a pH of about 5.5 to about 6.5. In another embodiment, the acidic conditions comprise a pH of about 6.

In one embodiment, the cells are cultured under acidic conditions for a timepoint sufficient to induce upregulation of VEGF-R2. In another embodiment, the cells are cultured under acidic conditions for a timepoint sufficient to induce upregulation of TEM-1. In one embodiment, the cells are cultured under acidic conditions for a timepoint sufficient to induce upregulation of CD-105, CD-73, and/or CD 39. In yet another embodiment, the cells are cultured under acidic conditions for a timepoint sufficient to induce upregulation of HLA-G. In still another embodiment, the cells are cultured under acidic conditions for a timepoint sufficient to induce upregulation of FAS-L.

In one embodiment, the endothelial progenitor cells or products thereof are made to resemble tumor endothelium by culture in a media depleted of amino acids in order to achieve GCN2 kinase. In yet another embodiment, the endothelial progenitor cells or products thereof are made to resemble tumor endothelium by culture in a media containing adenosine.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure As it will be realized, the present disclosure is capable of other different embodiments and its several details are capable of modifications in various obvious embodiments all without departing from, or limiting, the scope herein. Accordingly, descriptions will be regarded as illustrative in nature and not as restrictive.

### DETAILED DESCRIPTION OF THE PREFERRED EMBODIMENTS

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where the event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included herein.

The present disclosure is directed to a method and system of utilizing endothelial progenitor cells and products derived from the endothelial progenitor cells as a cancer vaccine which selectively induces immunity towards tumor vasculature and not healthy, non-malignant, vasculature. In one embodiment, the invention teaches the utilization of culture conditions which mimic the tumor microenvironment as a means of creating a cellular population that resembles tumor endothelial cells. Culture conditions include the growth of endothelial progenitor cells in acidic conditions which resemble the tumor microenvironment. Numerous papers have characterized the acidic conditions in the tumor microenvironment and are incorporated by reference. Interestingly, tumor acidic conditions are believed to be associated with resistance to immunotherapy. In a recent study it was shown that an acidic pH environment blocked T-cell activation and limited glycolysis in vitro. IFNγ release blocked by acidic pH did not occur at the level of steady-state mRNA, implying that the effect of acidity was posttranslational. Acidification did not affect cytoplasmic pH, suggesting that signals transduced by external acidity were likely mediated by specific acid-sensing receptors, four of which are expressed by T cells. Notably, neutralizing tumor acidity with bicarbonate monotherapy impaired the growth of some cancer types in mice where it was associated with increased T-cell infiltration. Furthermore, combining bicarbonate therapy with anti-CTLA-4, anti-PD1, or adoptive T-cell transfer improved antitumor responses in multiple models, including cures in some subjects. In one embodiment of the invention, endothelial progenitor cells, or products thereof, are cultured under conditions in GCN2 kinase is activated, the conditions include culture in the presence of uncharged tRNA, tryptophan deprivation, arginine deprivation, asparagine deprivation, and glutamine deprivation.

In one embodiment, a therapeutic vaccine is provided targeting tumor endothelial cells based on immunization with a composition resembling tumor endothelial cells. Of particular use, the invention discloses the use of tumor endothelial immunization prior to chemotherapy administration in order to reduce chemotherapy associated neovascularization. A recent study demonstrated that adriamycin or paclitaxel, first-line chemotherapy agent, induced breast cancer cells to generate morphological, phenotypical and functional features of endothelial cells in vitro. In xenografts models, challenges from adriamycin or paclitaxel induced cancer cells to generate the majority of microvessels. Importantly, in breast cancer specimens from patients with neoadjuvant anthracycline-based or taxane-based chemotherapy, tumor-derived endothelial microvessels, lined by EGFR-amplified or/and TP53+-CD31+ endothelial cells, was significantly higher in patients with progressive or stable disease (PD/SD) than in those with a partial or complete response (PR/CR) [52]. In another embodiment, the concurrent use of immunization targeting tumor endothelium together with inhibition of Notch signaling is provided. In another embodiment the concurrent use of VEGFR3 inhibition together with immunization against tumor endothelial cells is provided.

It is known that the tumor microenvironment possesses various mechanical properties which effect the tumor cells and tumor endothelial cells, specifically, the extracellular matrix (ECM) around tumors is found to stiffen progressively in a variety of human cancer types. Perhaps the best example is in breast cancer, which is often first detected by direct palpitation due to its increased stiffness compared to surrounding tissues. The presence of a fibrotic focus, which is an accumulation of collagen and fibroblasts, is correlated with an increase in metastatic disease and a decrease in recurrence-free survival in patients.

Boyd et al. undertook two studies of twins to determine the proportion of the residual variation in the percentage of density measured by mammography that can be explained by unmeasured additive genetic factors (heritability). A total of 353 pairs of monozygotic twins and 246 pairs of dizygotic twins were recruited from the Australian Twin Registry, and 218 pairs of monozygotic twins and 134 pairs of dizygotic twins were recruited in Canada and the United States. Information on putative determinants of breast density was obtained by questionnaire. Mammograms were digitized, randomly ordered, and read by a blinded investigator. After adjustment for age and measured covariates, the correlation coefficient for the percentage of dense tissue was 0.61 for monozygotic pairs in Australia, 0.67 for monozygotic pairs in North America, 0.25 for dizygotic pairs in Australia, and 0.27 for dizygotic pairs in North America. According to the classic twin model, heritability (the proportion of variants attributable to additive genetic factors) accounted for 60 percent of the variation in density (95 percent confidence interval, 54 to 66) in Australian twins, 67 percent (95 percent confidence interval, 59 to 75) in North American twins, and 63 percent (95 percent confidence interval, 59 to 67) in all twins studied. Confirmation that the mechanical stiffness associated with fibrosis correlates with poor prognosis was a study examining fibrotic foci (FF) in patients with ductal invasive carcinoma (DIC). The authors examined the presence of FF on the outcomes of 439 patients with IDCs to confirm the prognostic significance of FF, by the multivariate analysis, employing the Cox proportional hazard regression model, as compared with well-known clinicopathological parameters.

We also precisely evaluated the prognostic significance of FF from the viewpoint of FF characteristics. The present study demonstrated that the presence of FF is a very useful parameter predicting tumor recurrence (TR), as well as initial distant organ metastasis (IDOM), in lymph node-negative IDCs (P=0.024 and P=0.026) and in IDCs positive for either or both estrogen receptor (ER) or progesterone receptor (PR) (P=0.007 and P=0.015), respectively. In addition, FF of >8 mm in diameter was found to be an independent prognostic parameter for TR and IDOM in lymph node-negative patients and patients with IDC positive for either or both ER or PR (P=0.005 and P=0.018). We conclude that the presence of FF is a very important histologic prognostic parameter for patients with IDCs of the breast. A similar study 450 cases of breast carcinomas were evaluated for the presence of FF and its association with clinicopathologic parameters and biomarkers. FF was found in 18.7% of all consecutive cases. FF was associated positively with infiltrative margins (p=0.03) but negatively with extensive in situ component (p<0.001) and lymphocytic infiltration (p<0.001). It was positively associated with estrogen receptor (p=0.007) but negatively with human epidermal growth factor receptor 2 (HER2; p=0.001), epidermal growth factor receptor (p=0.021), Ki-67 (p=0.001), and c-kit (p=0.009). Concomitantly, FF was seen more commonly in luminal A cancers (p<0.001) but less so in luminal B (p=0.045) and HER2-overexpressing cancers (p=0.011). Analysis on patient outcome (median 41 months, range 1-69 months) indicated that FF was an independent poor prognostic factor for disease-free survival (hazard ratio=2.57; 95% confidence interval=1.267-5.214, p=0.009), particularly in the luminal B subtype. The findings suggested that FF is associated with specific tumor morphology of an infiltrative, stellate pattern (typical invasive ductal carcinoma-not otherwise specified) rather than round, cellular mass with intense lymphocytic infiltrate (basal-like breast cancers). The poor prognostic implication of FF is additional and independent of other adverse prognostic indicators.

In order to generate an environment similar to the tumor microenvironment in terms of stiffness of fibrotic tissue, one approach involves crosslinking of ECM, the ECM includes collagen, fibrin, fibronectin and hyaluronic acid. In one specific embodiment, collagen is extracted from an extracellular matrix source, the sources may include decellularized tissues. In one embodiment ECM may be extracted from tumor tissues. In another embodiment, it may be extracted from placental sources. Means of extracting ECM from placental sources are known. For example, placenta may be manipulated to obtain the desired portion, e.g., to obtain a desired placental circulatory unit (e.g., a cotyledon) before the portion of the placenta is further processed (e.g., processed as described herein, e.g., decellularized). In certain embodiments, when only a portion of a placenta is used in the generation of the organoids described herein, the entire placenta is processed as desired (e.g., decellularized as described below), followed by isolation of the specific portion of the placenta to be used (e.g., by cutting or stamping out the desired portion of the placenta from the whole processed placenta). Once the placenta is prepared as above, and optionally perfused, it is decellularized in such a manner as to preserve the native structure of the placental vasculature, e.g., leave the placental vasculature substantially intact. As used herein, “substantially intact” means that the placental vasculature remaining after decellularization retains all, or most, of the gross structure of the placental vasculature prior to decellularization. In certain embodiments, the placental vasculature is capable of being re-seeded, e.g., with vascular endothelial cells or other cells, specifically tumor cell lines so as to recreate the tumor vasculature.

The Placental tissue may be sterilized, e.g., by incubation in a sterile buffered nutrient solution containing antimicrobial agents, for example an antibacterial, an antifungal, and/or a sterilant compatible with the transplant tissue. The sterilized placental tissue may then be cryopreserved for further processing at a later time or may immediately be further processed according to the next steps of this process including a later cryopreservation of the tissue matrix or other tissue products of the process. Means of decellularizing tissue including physical, chemical, and biochemical methods. See, e.g. U.S. Pat. No. 5,192,312 (Orton) which is incorporated herein by reference. Such methods may be employed in accordance with the process described herein. However, the decellularization technique employed preferably does not result in gross disruption of the anatomy of the placental tissue or substantially alter the biomechanical properties of its structural elements, and preferably leaves the placental vasculature substantially intact. In certain embodiments, the treatment of the placental tissue to produce a decellularized tissue matrix does not leave a cytotoxic environment that mitigates against subsequent repopulation of the matrix with cells that are allogeneic or autologous to the recipient. As used herein, cells and tissues that are “allogeneic” to the recipient are those that originate with or are derived from a donor of the same species as a recipient of the placental vascular scaffold, and “autologous” cells or tissues are those that originate with or are derived from a recipient of the placental vascular scaffold. In one embodiment, the placental tissue is cryopreserved in the presence of one or more cryoprotectants. Colloid-forming materials may be added during freeze-thaw cycles to alter ice formation patterns in the tissue. For example, polyvinylpyrrolidone (10% w/v) and dialyzed hydroxyethyl starch (10% w/v) may be added to standard cryopreservation solutions (DMEM, 10% DMSO, 10% fetal bovine serum) to reduce extracellular ice formation while permitting formation of intracellular ice. In some embodiments, the placental tissue is decellularized using detergents or combinations thereof, for example, a nonionic detergent, e.g., Triton X-100, and an anionic detergent, e.g., sodium dodecyl sulfate, may disrupt cell membranes and aid in the removal of cellular debris from tissue. Preferably, residual detergent in the decellularized tissue matrix is removed, e.g., by washing with a buffer solution, so as to avoid interference with the later repopulating of the tissue matrix with viable cells.

In one embodiment, the means of decellularization is performed by the administration of a solution effective to lyse native placental cells. Preferably, the solution is an aqueous hypotonic or low ionic strength solution formulated to effectively lyse the cells. In certain embodiments, the aqueous hypotonic solution is, e.g. deionized water or an aqueous hypotonic buffer. In specific embodiments, the aqueous hypotonic buffer contains one or more additives that provide sub-optimal conditions for the activity of one or more proteases, for example collagenase, which may be released as a result of cellular lysis. Additives such as metal ion chelators, for example 1,10-phenanthroline and ethylenediaminetetraacetic acid (EDTA), create an environment unfavorable to many proteolytic enzymes. In other embodiments, the hypotonic lysis solution is formulated to eliminate or limit the amount of divalent cations, e.g., calcium and/or zinc ions, available in solution, which would, in turn, reduce the activity of proteases dependent on such ions.

It is important to prevent formation of viscous liquids during the decellularlization process, accordingly, in some embodiments, decellularization of placental tissue includes treatment of the tissue with one or more nucleases, e.g., effective to inhibit cellular metabolism, protein production and cell division without degrading the underlying collagen matrix. Nucleases that can be used for digestion of native cell DNA and RNA include either or both of exonucleases or endonucleases. Suitable nucleases for decellularization are commercially available. For example, it is known that exonucleases that effectively inhibit cellular activity include DNAase I (SIGMA Chemical Company, St. Louis, Mo.) and RNAase A (SIGMA Chemical Company, St. Louis, Mo.) and endonucleases that effectively inhibit cellular activity include EcoRI (SIGMA Chemical Company, St. Louis, Mo.) and Hind III (SIGMA Chemical Company, St. Louis, Mo.). For the practice of the invention, selected nucleases may be contained in a physiological buffer solution which contains ions that are optimal for the activity of the nuclease, e.g., magnesium salts or calcium salts. It is also preferred that the ionic concentration of the buffered solution, the treatment temperature and the length of treatment are selected to assure the desired level of effective nuclease activity. The buffer is preferably hypotonic to promote access of the nucleases to cell interiors. In certain embodiments, the one or more nucleases comprise DNAase I and RNAase A. Preferably, the nuclease degradation solution contains about 0.1 microgram/mL to about 50 microgram/mL, or about 10 microgram/mL, of the nuclease DNAase I, and about 0.1 microgram/mL to about 10 microgram/mL, preferably about 1.0 microgram/mL, of RNAase A. The placental tissue may be decellularized by application of the foregoing enzymes at a temperature of about 20° C. to 38° C., preferably at about 37° C., e.g., for about 30 minutes to 6 hours.

It is known in the art that the process of decellularization is associated with creation of tissue debris, therefore the placental tissue matrix in certain embodiments is washed in a wash solution to assure removal of cell debris which may include cellular protein, cellular lipids, and cellular nucleic acid, as well as any extracellular debris. Removal of this cellular and extracellular debris reduces the likelihood of the transplant tissue matrix eliciting an adverse immune response from the recipient upon implant. For example, the tissue may be washed one or more times with a wash solution, wherein the wash solution is, e.g., PBS or Hanks' Balanced Salt Solution (HBSS). The composition of the balanced salt solution wash, and the conditions under which it is applied to the transplant tissue matrix may be selected to diminish or eliminate the activity of proteases or nucleases utilized during the decellularization process. In specific embodiments, the wash solution does not contain magnesium or calcium, e.g. magnesium salts or calcium salts, and the washing process proceeds at a temperature of between about 2° C. and 42° C., e.g., 4° C. most preferable. The transplant tissue matrix may be washed, e.g., incubated in the balanced salt wash solution for up to 1, 2, 3, 4, 5, 6, 7, 8, 9, 10, 11 or 12 days, e.g., with changes in wash solution every −13 days. Optionally, an antibacterial, an antifungal or a sterilant or a combination thereof, may be included in the wash solution to protect the transplant tissue matrix from contamination with environmental pathogens. Washing may be performed by soaking the placental tissue with or without mild agitation.

To allow for large scale production, it may not be feasible to seed the tissue the same day that the cells are added. Accordingly, the placental tissue matrix, once decellularized, can be preserved by cryopreservation. Techniques of cryopreservation of tissue are well known in the art. See, e.g., Brockbank, K. G. M., “Basic Principles of Viable Tissue Preservation,” In: Transplantation Techniques and Use of Cryopreserved Allograft Cardiac Valves and Vascular Tissue, D. R. Clarke (ed.), Adams Publishing Group, Ltd., Boston. pp 9-23 (discussing cryopreservation of tissues and organs). The tissue matrix, whether or not having been cryopreserved, in certain embodiments is treated to enhance the adhesion and inward migration of the allogeneic or autologous cells, in vitro, which will be used to repopulate the transplant tissue. In certain embodiments, attachment of autologous or allogeneic cells to decellularized placental vascular scaffold may be increased, e.g., by contacting the placental vascular scaffold with serum (human or fetal bovine, maximal binding with 1% serum) and/or purified fibronectin, e.g., in culture medium in which the decellularized placental vascular scaffold is placed, e.g., in preparation for repopulation with allogeneic or autologous cells. Each of the two homologous subunits of fibronectin has two cell recognition regions, including one comprising the Arg-Gly-Asp (RGD) sequence. A second site, binding glycosaminoglycans, acts synergistically and appears to stabilize the fibronectin-cell interactions mediated by the RGD sequence. Additionally, platelet rich plasma, or platelet lysate may be utilized.

As such, in a specific embodiment, the decellularized placental vascular scaffold is contacted with both fibronectin and a glycosaminoglycan, e.g., heparin, for a period effective for binding of the fibronectin to surfaces of the placental vascular scaffold to be repopulated with allogeneic or autologous cells. The fibronectin, and optionally glycosaminoglycan, can be included within a physiologically-acceptable buffer or culture medium, e.g., sodium phosphate/glycerin/bovine serum albumin and Dulbecco's Modified Eagle's Medium (DMEM) (e.g., GIBCO). The buffer or culture medium is preferably maintained at a physiologically acceptable pH, e.g., about 6.8 to 7.6. Fibronectin may be obtained from human blood, processed to limit contamination with virus, or may be obtained from commercial sources. The concentration of fibronectin and/or glycoprotein may range from about 1 microgram/mL to about 100 microgram/mL, e.g., about 10 microgram/mL. The preferred weight ratio of fibronectin to heparin is about 100:1 to about 1:100, or about 10:1 to about 1:10, e.g., 10:1 fibronectin:glycosaminoglycan, e.g. heparin. The decellularized placental vascular scaffold may be contacted with, e.g., treated with, one or more compositions that act, e.g., to enhance cell chemotaxis, increasing the rate of directional movement along a concentration gradient of the substance in solution. With respect to fibroblast cells, fibroblast growth factor, platelet-derived growth factor, transforming growth factor-beta (TGF-β), fibrillar collagens, collagen fragments, and fibronectin are chemotactic. Crosslinking may be performed by reduction with standardized NaB3H4, hydrolyzed with 6N HCl and subjected to amino acid and collagen crosslink analyses. The reducible crosslinks, dehydro-dihydroxylysinonorleucine (deH-DHLNL) and deH-hydroxylysinonorleucine (deH-HLNL), their ketoamines, were identified as their reduced forms, i.e. DHLNL and HLNL. The crosslinks analyzed (reducible and nonreducible) were quantified as moles/mole of collagen based on the hydroxyproline value of 300 residues per collagen molecule. LOX activity was measured in cell supernatant and plasma

The generation of cells for immunogenic purposes to induce antitumor immunity through attacking tumor endothelium may be grown on a matrix as described above, with the relative stiffness of the matrix to resemble that found in cancer microenvironment. Evaluation of the stiffness of the matrix and manipulation of the matrix may be assessed as described in the following reference.

It is known that the tumor stroma is characterized by ECM remodeling and stiffening and tissue stiffness has been exploited to detect cancer. ECM stiffness enhances cell growth and survival and promotes migration, and ECM rigidity disrupts tissue morphogenesis by increasing cell tension. Reducing cell tension repressed the malignant behavior of mammary epithelial cells (MECs) and normalized the behavior of breast cancer cells in culture. Accordingly, within the practice of the current invention the use of existing techniques is utilized to induce an in vitro 3 dimensional system to generate a tumor-like environment in vitro in order to allow endothelial progenitor cells or endothelial cells derived from them to resemble tumor endothelial cells, which subsequently are used for immunization.

Collagen is the most abundant ECM scaffolding protein in the stroma and contributes significantly to the tensile strength of tissue. Collagen metabolism is deregulated in cancer, where increased collagen expression, elevated deposition, altered organization, and enhanced matrix metalloproteinase (MMP) activity and collagen turnover have been implicated in tumor progression. MMP-mediated collagen remodeling can create space for cells to migrate, produce substrate cleavage fragments with independent biological activity, modify adhesion to regulate tissue architecture, and activate, deactivate, or alter the activity of signaling molecules. Although high levels of MMPs correlate with poor prognosis in cancer patients, and modulating MMP activity changes tumor phenotype, MMP inhibitors failed clinically, indicating other ECM remodeling parameters regulate malignancy. In some embodiments the MMPs are utilized as modifiers of the cellular culture to endow endothelial cells with properties similar to tumor associated endothelial cells.

“Marker” and “Biomarker” are used interchangeably to refer to a gene expression product that is differentially present in a samples taken from two different subjects, e.g., from a test subject or patient having (a risk of developing) an ischemic event, compared to a comparable sample taken from a control subject (e.g., a subject not having (a risk of developing) an ischemic event; a normal or healthy subject). Alternatively, the terms refer to a gene expression product that is differentially present in a population of cells relative to another population of cells.

The phrase “differentially present” refers to differences in the quantity or frequency (incidence of occurrence) of a marker present in a sample taken from a test subject as compared to a control subject. For example, a marker can be a gene expression product that is present at an elevated level or at a decreased level in blood samples of a risk subjects compared to samples from control subjects. Alternatively, a marker can be a gene expression product that is detected at a higher frequency or at a lower frequency in samples of blood from risk subjects compared to samples from control subjects.

A gene expression product is “differentially present” between two samples if the amount of the gene expression product in one sample is statistically significantly different from the amount of the gene expression product in the other sample. For example, a gene expression product is differentially present between two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

As used herein, the terms “antibody” and “antibodies” refer to monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a polypeptide antigen encoded by a gene comprised in the genomic regions or affected by genetic transformations in the genomic regions listed in Table 1. The immunoglobulin molecules of the invention can be of any type (e.g., IgG, IgE, IgM, IgD, IgA and IgY), class (e.g., IgG.sub.1, IgG.sub.2, IgG.sub.3, IgG.sub.4, IgA.sub.1 and IgA.sub.2) or subclass of immunoglobulin molecule.

“Immunoassay” is an assay that uses an antibody to specifically bind an antigen (e.g., a marker). The immunoassay is characterized by the use of specific binding properties of a particular antibody to isolate, target, and/or quantify the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The phrase “specifically (or selectively) binds” when referring to an antibody, or “specifically (or selectively) immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

The terms “affecting the expression” and “modulating the expression” of a protein or gene, as used herein, should be understood as regulating, controlling, blocking, inhibiting, stimulating, enhancing, activating, mimicking, bypassing, correcting, removing, and/or substituting the expression, in more general terms, intervening in the expression, for instance by affecting the expression of a gene encoding that protein.

In one embodiment, EPCs refer to endothelial colony-forming cells (ECFCs) and their progenitor cell capacities were characterized as described (Wu, Y et al., J Thromb Haemost, 2010; 8:185-193; Wang, H et al., Circulation research, 2004; 94:843 and Stellos, K et al., Eur Heart J., 2009; 30:584-593). Briefly, human blood was collected from healthy volunteer donors. All volunteers had no risk factors of CVD including hypertension, diabetes, smoking, positive family history of premature CVD and hypercholesterolemia, and were all free of wounds, ulcers, retinopathy, recent surgery, inflammatory, malignant diseases, and medications that may influence EPC kinetics. After dilution with HBSS (1:1), blood was overlaid onto Histopaque 1077 (Sigma-Aldrich Co. LLC, St. Louis, Mo.) in the ratio of 1:1 and centrifuged at 740 g for 30 minutes at room temperature. Buffy coat MNCs were collected and centrifuged at 700 g for 10 minutes at room temperature. MNCs were cultured in collagen type I (BD Bioscience, San Diego) (50 m/ml)-coated dishes with EBM2 basal medium (Lonza Inc., Allendale, N.J.) plus standard EGM-2 SingleQuotes (Lonza Inc., Allendale, N.J.) that includes 2% fetal bovine serum (FBS), EGF (20 ng/ml), hydrocortisone (1 .mu.g/ml), bovine brain extract (12 .mu.g/m1), gentamycin (50 m/ml), amphotericin B (50 ng/ml), and epidermal growth factor (10 ng/ml). Colonies appeared between 5 and 22 days of culture were identified as a well-circumscribed monolayer of cobblestone-appearing cells. ECFCs with endothelial lineage markers expression, robust proliferative potential, colony-forming, and vessel-forming activity in vitro are defined as EPCs as described (Wang, H et al., Circulation research, 2004; 94:843 and Stellos, K et al., Eur Heart J., 2009; 30:584-593). Passage 4 to 6 EPCs were used for experiments. For a brief characterization, endothelial phagocytosis function was confirmed by incubating EPC in 4-well chamber slide with 1,1-dioctadecyl-3,3,3,3-tetramethylindocarbocyanine (DiI)-labeled acetylated low-density lipoprotein (acLDL) (Biomedical Technologies, Inc., Stoughton, Mass.) (5 m/ml) at 37.degree. C. for 1 h, washed 3 times for 15 min in PBS, and then fixed with 2% paraformaldehyde for 10 min. Cells were then incubated with FITC conjugated UEA-1 (Ulex europaeus agglutinin) (10 m/ml) (Sigma-Aldrich Corporation, St. Louis, Mo.) for 1 h at room temperature, which is capable of binding with glycoproteins on the cell membrane to allow visualization of the entire cell. Cell integrity was examined by nuclear staining with DAPI (100 ng/ml). After staining, cells are imaged with high-power fields under an inverted fluorescent microscope (Axiovert 200, Carl Zeiss, Thornwood, N.Y.) at 200.times. magnification and quantified using Image J software.

The potential of using the tumor vasculature as a target is enticing, however previous studies have not utilized polyvalent antigenic entities, or in the cases where they have, such as in cellular vaccines, the cells where either not made to be immunogenic, nor are the cells grown under conditions which induce replicate the tumor microenvironment. The following examples are provided to allow the practitioner of the invention to ascertain various immunization regimens, adjuvants, and combinations. The invention teaches means of “focusing” an immune response subsequent to immunization with a polyvalent cancer vaccine targeting tumor associated blood vessels. In one embodiment, patients suffering from cancer are immunized with ValloVax [59], or a vaccine composition similar to tumor endothelial cells. Active immunization against tumor endothelium by vaccinating against proliferating endothelium or markers found on tumor endothelium has provided promising preclinical data. Specifically, in animal models it has been reported that immunization to antigens specifically found on tumor vasculature can lead to tumor regression. Studies have been reported using the following antigens: survivin, endosialin, and xenogeneic FGF2R, VEGF, VEGF-R2, MMP-2, and endoglin. Human trials have been conducted utilizing human umbilical vein endothelial (HUVEC) cells as tumor antigens, with responses being reported in patients. In one report describing a 17-patient trial, Tanaka et al demonstrated that HUVEC vaccine therapy significantly prolonged tumor doubling time and inhibited tumor growth in patients with recurrent glioblastoma, inducing both cellular and humoral responses against the tumor vasculature without any adverse events or noticeable toxicities.

The invention provides the use of tissue or circulating EPC as a substrate for transformation into an immunogenic cell population resembling tumor associated endothelial cells. The EPC is an undifferentiated cell that can be induced to proliferate using the methods of the present invention. The EPC is capable of self-maintenance, such that with each cell division, at least one daughter cell will also be an EPC cell. EPCs are capable of being expanded 100, 250, 500, 1000, 2000, 3000, 4000, 5000 or more fold. Phenotyping of EPCs reveals that these cells express the committed hematopoietic marker CD45. Additionally, an EPC is immunoreactive for VEGFR-2. The EPC is a multipotent progenitor cell. By multipotent progenitor cell is meant that the cell is capable of differentiating into more than one cell type. For example, the cell is capable of differentiating into an endothelial cell or a smooth muscle cell. Vascular endothelial growth factor (VEGF) acts through specific tyrosine kinase receptors that includes VEGFR-1 (flt-1) and VEGFR-2 (flk-1/KDR) and VEGFR-3/Flt-4 which convey signals that are essential for embryonic angiogenesis, cancer angiognesis and hematopoiesis. While VEGF binds to all three receptors, most biological functions are mediated via VEGFR-2 and the role of VEGFR-1 is currently unknown. VEGFR3/Flt4 signaling is known to be important for the development of lymphatic endothelial cells and VEGFR3 signaling may confer lymphatic endothelial-like phenotypes to endothelial cells. VEGFRs relay signals for processes essential in stimulation of vessel growth, vasorelaxation, induction of vascular permeability, endothelial cell migration, proliferation and survival. Endothelial cells express all different VEGF-Rs. During embryogenesis, it has been reported that a single progenitor cell, the hemangioblast can give rise to both the hematopoietic and vascular systems. In the process of tumor angiogenesis, VEGF plays a fundamental role in promoting malignant and leaky angiogenesis.

It is known that VEGF is stimulated in part by hypoxia, and by activation of SDF-1 through HIF-1 alpha. In one embodiment endothelial progenitor cells are treated with a combination of VEGF and other tumor associated factors. In order to induce the generation of endothelial cells the resemble tumor endothelial cells, culture under conditions that stimulate HIF-1 alpha are used. In one embodiment the invention discloses means of modifying through culture endothelial cells. One type of culture condition involves growth of cells under hypoxia. Numerous means of culturing cells in hypoxia are known in the art and are described in the following references. Other means of inducing cellular signaling mimicking hypoxia include treatment with tissue factor, or tissue factor activating compounds. Other approaches to activating these pathways include treatment with LRG-1, culture with macrophages, treatment with CCL5, culture with lactic acid alone or in the presence of monocytes, culture in hyaluronic acid with stiffness similar to tumor associated stiffness, culture in isoflurane, culture in MUC1, and culture in cadminum.

Various sources of EPC can be used, the EPC can be either derived from placental sources, cord tissue, and bone marrow. EPCs can also be cultured in vitro to maintain a source of EPCs, or can be induced to produce further differentiated EPCs that can develop into a desired tissue. The cells of the invention can be obtained by mechanically and enzymatically dissociating cells from bone marrow, placental, adipose, or umbilical cord tissue. Mechanical dissociation can be brought about using methods that include, without limitation, chopping and/or mincing the tissue, and/or centrifugation and the like. Enzymatic dissociation of connective tissue and from cell-to-cell associations can be brought about by enzymes including, but not limited to, Blendzyme, DNAse I, collegenase and trypsin, or a cocktail of enzymes found to be effective in liberating cells from the bone marrow sample. The procedure for mechanically and enzymatically isolating a cell of the present invention should not be construed to be limited to the materials and techniques presented herein, but rather it will be recognized that these techniques are well-established and fall well within the scope of experimental optimization performed routinely in the art. In the case of bone marrow-derived EPCs of the invention are isolated from bone marrow. In the isolation of the cells of the invention, bone marrow can be obtained from any animal by any suitable method. A first step in any such method requires the isolation of bone marrow from the source animal. The animal can be alive or dead, so long as cells within bone marrow are viable. Typically, human bone marrow is obtained from a living donor, using well-recognized surgical protocols. The cells of the invention are present in the initially excised or extracted bone marrow, regardless of the method by which bone marrow is obtained. In another embodiment, bone marrow may be obtained from non-human animals. In one embodiment, a bone marrow is removed from the animal. In one embodiment, bone marrow is washed with a physiologically-compatible solution, such as phosphate buffer saline (PBS). The washing step consists of rinsing bone marrow with PBS, agitating the tissue, and allowing the tissue to settle. In one embodiment, bone marrow is dissociated. The dissociation can occur by enzyme degradation and neutralization. Alternatively, or in conjunction with such enzymatic treatment, other dissociation methods can be used such as mechanical agitation, sonic energy, or thermal energy.

In some instances, it may be desirable to further process the dissociated tissue. For example, the dissociated bone marrow can be filtered to isolate cells from other connective tissue. The extracted cells can be concentrated into a pellet. One method to concentrate the cells includes centrifugation, wherein the sample is centrifuged and the pellet retained. The pellet includes the bone marrow-derived EPCs of the invention.

In one embodiment, the cells are resuspended and can be washed (e.g. in PBS). Cells can be centrifuged and resuspended successive times to achieve a greater purity. In one embodiment, the cells extracted from bone marrow may be a heterogeneous population of cell which includes the bone marrow-derived EPCs of the invention. bone marrow-derived EPCs may be separated from other cells by methods that include, but are not limited to, cell sorting, size fractionation, granularity, density, molecularity, morphologically, and immunohistologically. In one embodiment, bone marrow-derived EPCs of the invention are separated from other cells by assaying the length of the telomere, as stem cells tend to have longer telomeres compared to differentiated cells. In another embodiment, bone marrow-derived EPCs of the invention are separated from other cells by assaying telomeric activity, as telomeric activity can serve as a stem-cell specific marker. In another embodiment, bone marrow-derived endothelial cells of the invention are separated from other cells immunohistochemically, for example, by panning, using magnetic beads, or affinity chromatography.

In one embodiment of the invention, cytotoxic lymphocytes that recognize tumor antigens or tumor endothelial antigens are cultured under conditions of the tumor microenvironment in order to condition the cytotoxic lymphocytes to become resistant to the immune suppressive effects of the tumor microenvironment. The generation of cytotoxic lymphocytes may be performed, in one embodiment by extracted 50 ml of peripheral blood from a cancer patient and peripheral blood mononuclear cells (PBMC) are isolated using the Ficoll Method. PBMC are subsequently resuspended in 10 ml AIM-V media and allowed to adhere onto a plastic surface for 2-4 hours. The adherent cells are then cultured at 37° C. in AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment the generated DC are used to stimulate T cell and NK cell tumoricidal activity by pulsing with autologous tumor lysate. Specifically, generated DC may be further purified from culture through use of flow cytometry sorting or magnetic activated cell sorting (MACS), or may be utilized as a semi-pure population. DC pulsed with tumor lysate may be added into the patient in need of therapy with the concept of stimulating NK and T cell activity in vivo, or in another embodiment may be incubated in vitro with a population of cells containing T cells and/or NK cells.

In one embodiment DC are exposed to agents capable of stimulating maturation in vitro and rendering them resistant to tumor derived inhibitory compounds such as arginase byproducts. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to the T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. The immune response of the patient treated with these cytotoxic cells is assessed utilizing a variety of antigens found in tumor endothelial cells. When cytotoxic or antibody, or antibody associated with complement fixation are recognized to be upregulated in the cancer patient, subsequent immunizations are performed utilizing peptides to induce a focusing of the immune response.

In one embodiment of the invention, immunization against antigens found on tumor endothelium, is induced utilizing a primary immunization utilizing a polyvalent combination. In one example tumor endothelial derived exosomes from the same patients may be collected, concentrated, and administered with adjuvant. Subsequent to immunization, specific immunity in a personalized manner is assessed. Immunity is assessed to specific antigens found on tumor endothelial cells, antigens include ROBO, VEGFR, VEGFR2, FGFR, TEM-1 and notch. Assessment of immunity is performed by quantifying reactivity of T cells or B cells in response to protein antigens or derivatives thereof, derivatives including peptide antigens or other antigenic epitopes. Responses may be assessed in terms of proliferative responses, cytokine release, antibody responses, or generation of cytotoxic T cells. Methods of assessing the responses are well known in the art. In a preferred embodiment, antibody responses are assessed to a panel of tumor endothelium associated proteins subsequent to immunization of patient. Antibody responses are utilized to guide which peptides will be utilized for prior immunization. For example, if a patient is immunized with ValloVax on a weekly basis, the subsequent assessment of antibody responses is performed at approximately 1-3 months after initiation of immunization. Protocols for immunization with ValloVax include weekly, biweekly, or monthly. Assessment of antibody responses is performed utilizing standard enzyme linked immunosorbent (ELISA) assay. Assessment of antibodies is performed, in one embodiment of the invention, against proteins associated with tumor endothelium such as ROBO, VEGFR, VEGFR2, FGFR, TEM-1 and notch. In patients in which a higher antibody responses is observed against VEGFR2, as an example, immunization with VEGFR2 antigenic epitopes is performed to enhance such specific immune response. In patients in which after ValloVax immunization possess an elevated antibody response to TEM-1, immunization with TEM-1 is performed. In patients in which antibodies significantly increase to numerous antigens, multiple peptide or antigenic combinations are utilized.

EPCs can be cultured and, if desired, assayed for number and viability, to assess the yield. In one embodiment, the stem cells are cultured without differentiation using standard cell culture media (e.g., DMEM, typically supplemented with 5-15% (e.g., 10%) serum (e.g., fetal bovine serum, horse serum, etc.). In one embodiment, the stem cells are passaged at least five times in such medium without differentiating, while still retaining their developmental phenotype. In one embodiment, the stem cells are passaged at least 10 times (e.g., at least 15 times or even at least 20 times) while retaining potency.

EPCs can be separated by phenotypic identification, to identify those cells that have two or more of the aforementioned developmental lineages. In one embodiment, all cells extracted from bone marrow are cultured. To phenotypically separate the bone marrow-derived EPCs from the other cells of bone marrow, the cells are plated at a desired density, such as between about 100 cells/cm2 to about 100,000 cells/cm2 (such as about 500 cells/cm2 to about 50,000 cells/cm2, or, more particularly, between about 1,000 cells/cm2 to about 20,000 cells/cm2).

In one embodiment the extracted cells of placental tissue is plated at a lower density (e.g., about 300 cells/cm2) to facilitate the clonal isolation of the placental EPCs. For example, after a few days, EPCs plated at such densities will proliferate (expand) into a clonal population of EPCs. Such EPCs can be used to clone and expand a clonal population, using a suitable method for cloning cell populations. The cloning and expanding methods include cultures of cells, or small aggregates of cells, physically picking and seeding into a separate plate (such as the well of a multi-well plate). Alternatively, the stem cells can be subcloned onto a multi-well plate at a statistical ratio for facilitating placing a single cell into each well (e.g., from about 0.1 to about 1 cell/well or even about 0.25 to about 0.5 cells/well, such as 0.5 cells/well).

The EPCs can be cloned by plating them at low density (e.g., in a petri-dish or other suitable substrate) and isolating them from other cells using devices such as a cloning rings. Alternatively, where an irradiation source is available, clones can be obtained by permitting the cells to grow into a monolayer and then shielding one and irradiating the rest of cells within the monolayer. The surviving cell then will grow into a clonal population. Production of a clonal population can be expanded in any suitable culture medium, for example, an exemplary culture condition for cloning stem cells (such as the inventive stem cells or other stem cells) is about ⅔ F12 medium+20% serum (e.g. fetal bovine serum) and about ⅓ standard medium that has been conditioned with stromal cells, the relative proportions being determined volumetrically). In any event, whether clonal or not, the isolated EPCs can be cultured in a specific inducing medium to induce the EPCs to differentiate and express its potency. The EPCs have the potential rise to cells of mesodermal, ectodermal and endodermal lineage, and combinations thereof, most importantly the EPC can be used as a source of undifferentiated cells for transformation into tumor endothelial-like cells. Alternatively, the EPCs are cultured in a conditioned medium and induced to differentiate into a specific phenotype. Conditioned medium is medium which was cultured with a mature cell that provides cellular factors to the medium such as cytokines, growth factors, hormones, and extracellular matrix. For example, a medium that has been exposed to mature tumor endothelial cells. For co-culture, it may be desirable for the EPCs and the desired other cells to be co-cultured under conditions in which the two cell types are in contact. This can be achieved, for example, by seeding the cells as a heterogeneous population of cells onto a suitable culture substrate. Alternatively, the bone marrow-derived EPCs can first be grown to confluence, which will serve as a substrate for the second desired cells to be cultured within the conditioned medium. Second sets of cells include tumor cells, monocytes, fibroblasts, and other cells that are associated with the cancer microenvironment.

In some embodiments, culture of the immune effectors cells is performed after extracting from a patient that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used.

In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD40L, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG.

The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide.

Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects. The cells can be expanded in the presence of specific antigens associated with tumor endothelium and subsequently injected into the patient in need of treatment. Expansion with specific antigens includes coculture with proteins selected from a group comprising of: a) ROBO; b) VEGF-R2; c) FGF-R; d) CD105; e) TEM-1; and f) survivin. Other tumor endothelial specific or semi-specific antigens are known in the art that may be used.

For the practice of the invention, EPC may be isolated based on the in vitro culture of mononuclear cells on fibronectin- or gelatin-coated plates in the presence of angiogenic growth factors [99-116]. The cells may be isolated from peripheral blood, cord blood, digested placental cells, umbilical cord cells, and bone marrow. Isolated adherent cells that are low density lipoprotein (LDL) positive and exhibited lectin-binding ability are termed EPC. Although these cells promote angiogenesis in vivo, they have monocytic features and their angiogenicity is actually caused by their production of angiogenic factors, such as VEGF, hepatocyte growth factor (HGF), G-CSF and GM-CSF. Thus, while these LDL positive, lectin-binding cells do not directly form EC, they can modulate angiogenesis. These cells are useful for starting material to generated immunogenic cells similar to tumor endothelial cells. Human CD34+ EPC and CD14+ monocytes may be co-cultivated at different ratios, ranging from their physiological ratio in peripheral blood to an enriched ratio of CD34+ EPC (1:10, 1:100, and 1:1000). The CD34+ EPCs augment endothelial cell differentiation from CD14+ monocytic cells in vitro. The CD34+ EPC not only stimulate a higher proportion of endothelial cell-like clusters, but expression of the endothelial cell markers von Willebrand Factor (vWF) and VE-cadherin is higher in co-cultured cells than in mono-cultured CD14+ cells. Additionally, CD34+ EPC express pro-angiogenic genes such as EGF, HGF, VEGF-a, bFGF and IGF and IL-8. The addition of VEGF, HGF, bFGF and IGF to CD14+ mono-cultures ameliorates CFU formation, and thus EC outgrowth, from these colonies, increasing ability to generate cells similar to tumor vasculature. The present invention therefore provides methods and compositions for generating cells similar to tumor endothelial cells.

In embodiments of the present invention, the CD34+ EPCs and CD14+ monocytes are autologous, allogenic, or HLA non-compatible with the subject. The ratio of the purified CD34+ EPCs to purified CD14+ monocytes in the mixture can be, for example, from about 1:1 to about 1:10000. Alternatively, the ratio can be, in non-limiting examples, from about 1:50 to about 1:5,000, from about 1:100 to about 1:2,000, from about 1:500 to about 1:1,000, from about 1:10 to about 1:1000, from about 1:10 to about 1:1000. In another embodiment, a method for generating cells similar to tumor endothelial cells comprising obtaining placental cells from the subject, individually isolating purified CD34+ EPCs and purified CD14+ monocytes from placental cells, co-culturing the purified CD34+ EPCs and purified CD14+ monocytes in a culture medium for up to four weeks to yield a population of co-cultured EPCs and administering a therapeutically effective amount of the co-cultured EPCs to a treatment site, thereby inducing neovascularization in the injured tissue. The ratio of the purified CD34+ EPCs to purified CD14+ monocytes in the co-culture can be, for example, from about 1:1 to about 1:10000. Alternatively, the ratio can be, in non-limiting examples, from about 1:50 to about 1:5,000, from about 1:100 to about 1:2,000 or from about 1:500 to about 1:1,000, from about 1:10 to about 1:1000, from about 1:10 to about 1:1000.

Purified CD34+EPCs and purified CD14+monocytes are cultured under conditions favorable to survival of EPCs. In one embodiment, the culture medium contains one or more than one growth factor selected from the group consisting of vascular endothelial growth factor and basic fibroblast growth factor. In one non-limiting example, the culture medium (GMX) consists of RPMI 1640, supplemented with 20% Fetal Calf Serum, 5 U/mL heparin, 2 mM L-glutamine 1% Penicillin/Streptomycin, endothelial cell growth factor (5 .mu.g/mL), VEGF-A (1 ng/mL) and bFGF (10 ng/mL). The CD34+ and CD14+ cells can be co-cultured in medium containing serum from other sources including, but not limited to, human serum, autologous human serum, etc. The cells are co-cultured for up to about four weeks, alternatively, for about one week, for about two weeks or for about three weeks. At the end of the co-culture period the cells are washed and prepared for administration to a patient or cryopreserved according to established protocols known to persons of ordinary skill in the art.

The therapeutically effective amount of the co-cultured EPCs or mixture of purified CD34+ EPCs and purified CD14+ monocytes can be suspended in a pharmaceutically acceptable carrier or excipient. Such carriers include, but are not limited to, basal culture medium plus 1% serum albumin, saline, buffered saline, dextrose, water, biodegradable biocompatible matrices, and combinations thereof. Examples of biodegradable biocompatible matrices include, but are not limited to, solubilized basement membrane, autologous platelet gel, collagen gels or collagenous substrates based on elastin, fibronectin, laminin, extracellular matrix and fibrillar proteins, alginates, chitosans, and synthetic compositions such poly lactic acid, poly glycolic acid, polyethylene oxide, polyethylene glycol, etc. The formulation should suit the mode of administration. Accordingly, the invention provides a use of endothelial producing cells, such as co-cultured EPCs or a mixture of purified CD34+ EPCs and purified CD14+ monocytes, for the manufacture of a medicament to induce immunity to tumor endothelial cells. In some embodiments, the medicament further comprises growth factors, chemokines or cytokines.

The present invention provides isolated stem cell and endothelial progenitor cell populations derived from fetal vascular lobules of a hemochorial placenta, particularly a hemochorial placenta from a human. In one embodiment of the invention the stem and/or endothelial progenitor cells express CD144, CD105, and/or CD31, either immediately upon isolation or after culturing. In certain aspects, stem cells and/or endothelial progenitor cells of the invention do not express CD45. In one embodiment of the invention, the stem and/or endothelial progenitor cells express CD144, CD105, and CD31 but do not express do not express CD45.

In specific embodiments EPC are isolated with ability to form capillary-like tubules when plated on a Matrigel substrate and can take up DiI-acetylated-low-density-lipoprotein. In certain embodiments, the isolated EPC of the invention are prepared by homogenizing fetal vascular lobules from a full-term placenta; successively digesting the homogenized lobules with a preparation of about 2% collagenase, about 0.25% trypsin and about 0.1% DNAse, in tissue culture medium such as DMEM. The digestion product is then filtered to remove particulates, and mononuclear cells are obtained therefrom by density gradient centrifugation. The mononuclear cells can then be plated on collagen I-coated tissue culture plates and grown to confluency. Detached cells from the confluent plates are then sorted to obtain stem and/or progenitor cells that express of CD144 but lack of expression of CD45. In certain embodiments of the invention, the isolated stem and/or endothelial progenitor cells of the invention have the ability to differentiate into mesoderm, ectoderm and endoderm. In other embodiments of the invention, the isolated stem and/or endothelial progenitor cells of the invention have the ability to differentiate into at least one of mesoderm, ectoderm or endoderm.

In certain aspects, the isolated EPC can be expanded in vitro prior to administration. In other embodiments, the isolated stem or endothelial progenitor cells can be activated in vitro prior to administration. Activation can, for example, include treatment with at least one agent or condition capable of upregulating a CXCR-4 receptor, such as IL-1, IL-6, stem cell factor, flt-3L, hepatocyte growth factor, exposure to hypoxic conditions, a cytokine, a histone deacetylating agent, a DNA methyltransferase inhibitor, and an inhibitor of GSK-3 kinase.

In one embodiment EPC are a population of cells comprising cells having the surface marker CD44 [117], cells having the surface marker CD13, cells having the surface marker CD90, cells having the surface marker CD105, cells having the surface marker ABCG2, cells having the surface marker HLA 1, cells having the surface marker CD34, cells having the surface marker CD133, cells having the surface marker CD117, cells having the surface marker CD135, cells having the surface marker CXCR4, cells having the surface marker c-met, cells having the surface marker CD31, cells having the surface marker CD14, cells having the surface marker Mac-1, cells having the surface marker CD11, cells having the surface marker c-kit cells having the surface marker SH-2, cells having the surface marker VE-Cadherin, VEGFR and cells having the surface marker Tie-2s.

Once the cells of the invention have been established in culture, as described, they may be maintained or stored in “cell banks” comprising either continuous in vitro cultures of cells requiring regular transfer, or, preferably, cells which have been cryopreserved. Cryopreservation of cells of the invention may be carried out according to known methods, such as those described in Doyle et al., 1995, Cell and Tissue Culture. For example, but not by way of limitation, cells may be suspended in a “freeze medium” such as, for example, culture medium further comprising 15-20% FBS and 10% dimethylsulfoxide (DMSO), with or without 5-10% glycerol, at a density, for example, of about 4-10.times.10.sup.6 cells-ml.sup.-1. The cells are dispensed into glass or plastic ampoules (Nunc) that are then sealed and transferred to the freezing chamber of a programmable freezer. The optimal rate of freezing may be determined empirically. For example, a freezing program that gives a change in temperature of about −1.degree. C.-min.sup.-1 through the heat of fusion may be used. Once the ampoules have reached about −180.degree. C., they are transferred to a liquid nitrogen storage area. Cryopreserved cells can be stored for a period of years, though they should be checked at least every 5 years for maintenance of viability.

Exosomes are 30- to 100-nm diameter vesicles derived from a diverse range of cell types. DC-derived exosomes contain Ag-presenting, adhesion, and costimulatory molecules, which alone or in association with DCs can serve as a potent vaccine to stimulate strong CTL responses and induce antitumor immunity in different animal models [133]. For example, mature DCs pulsed with exosomes stimulate efficient CTL responses and antitumor immunity, and active CD4.sup.+ T cells following uptake of OVA-pulsed, DC-derived exosomes, can stimulate CD8.sup.+ T cell to proliferate and differentiate into central memory CD8.sup.+ CTLs, resulting in not only more efficient in vivo antitumor immunity and long-term CD8.sup.+ T-cell memory responses than OVA-pulsed dendritic cells, but also the ability to counteract CD4.sup.+ 25.sup.+ regulatory T-cell-mediated immune suppression. Thus, such exosome-targeted active CD4.sup.+ T cells may represent a novel and highly effective cancer vaccine targeting tumor endothelium.

In one study, researchers investigated the relative importance of exosomal major histocompatibility complex (MHC) class I for the induction of antigen-specific T cell responses and tumour protection. They showed that ovalbumin-loaded dendritic cell-derived exosomes from MHCI−/− mice induce antigen-specific T cells at the same magnitude as wild type exosomes. Furthermore, exosomes lacking MHC class I, as well as exosomes with both MHC class I and II mismatch, induced tumour infiltrating T cells and increased overall survival to the same extent as syngeneic exosomes in B16 melanoma. In conclusion, T cell responses are independent of exosomal MHC/peptide complexes if whole antigen is present. Generation of clinical grade exosomes has previously been demonstrated by others and these methods are applicable to the practice of the current invention.

In one embodiment DC are pulsed with tumor-endothelial-like cells generated from culture of endothelial progenitor cells under conditions mimicking tumor microenvironment and the cells are used a s a source of tumor antigen. In another embodiment, in vivo targeting of antigens to DCs represents a promising approach for DC-based vaccination, as it can bypass the laborious and expensive ex-vivo antigen loading and culturing, facilitating large-scale application of DC-based immunotherapy. More importantly, in vivo DC-targeted vaccination was reported to be more efficient in eliciting an anti-tumor immune response, and more effective in controlling tumor growth in animal models. One promising strategy developed for in-vivo targeted DC vaccination is to use engineered lentiviral vectors that specifically bind to the DC surface protein, DC-SIGN. Successful transduction of DCs in vivo by direct injection of a lentiviral vector encoding the human melanoma antigen, NY-ESO-1, under mouse dectin-2 gene promoter that restricted transgene expression to antigen-presenting cells, was also evidenced (Lopes et al., 2008, J. Virol. 82:86-95) by priming an NY-ESO-1-specific CD8.sup.+ T-cell response in HLA-A2 transgenic mice, and stimulating a CD4.sup.+ T-cell response to a newly identified NY-ESO-1 epitope presented by H2 I-A(b).

These results indicate that targeting antigen expression to DCs with lentiviral vectors can provide a safe and effective vaccine Targeting antigens to DCs via an antibody specific to a select DC cell surface marker is another approach for in-vivo targeted DC vaccination, as reported for mannose receptor (He et al., 2007, J. Immunol. 178:6259-6267; Ramakrishna et al., 2004, J. Immunol. 172(5), 2845-2852), CD205 (Trumpfheller et al., 2006, J. Exp. Med. 203:607-617; Gurer et al., 2008, Blood 112:1231-1239), DC-SIGN (Tacken et al., 2005, Blood 106:1278-1285), and LOX1 (Delneste et al., 2002, Immunity 17(3):353-362). In addition, the potential use of CD74 for in-vivo targeted DC vaccination is being explored by us. CD74 is a type-II integral membrane protein essential for proper MHC II folding and MHC II-CD74 complex targeting to endosomes (Stein et al., 2007, Clin. Cancer Res. 13:5556s-5563s; Matza et al., 2003, Trends Immunol. 24:264-268). CD74 expression is not restricted to DCs, but is in almost all antigen-presenting cells (Freudenthal et al., 1990, Proc. Natl. Acad. Sci. U.S.A. 87:7698-7702), including B cells, monocytes, and different DC subsets, such as blood myeloid DC1, myeloid DC2, plasmacytoid DC (Chen et al., 2008, Blood (ASH Annual Meeting Abstracts) 112: Abstract 2649), and follicular DCs (Clark et al., 1992, J. Immunol. 148:3327-3335). The broad expression of CD74 in APCs may offer some advantages over sole expression in myeloid DCs, because targeting of antigens to other APCs, like B cells, has been reported to break immune tolerance (Ding et al., 2008, Blood 112:2817-2825), and targeting to plasmacytoid DCs cross-presents antigens to naive CD8 T cells (Mouries et al., 2008, Blood 112:3713-3722). Furthermore, CD74 is also expressed in follicular DCs, a DC subset critical for antigen presentation to B cells (Clark et al., 1992, J. Immunol. 148:3327-3335). In one embodiment of the invention a cancer vaccine composition is produced by obtaining endothelial progenitor cells (EPC). The EPC may be identified by means by selecting for cells expressing genes, wherein genes are at least one gene selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lambl-1, LGMN, MMP3, Nos2, PAIL PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1). The EPC may be purified from a variety of sources, including peripheral blood, placental cells, cord blood, umbilical cord, adipose tissue and bone marrow.

In another embodiment, EPC are characterized by expression of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lambl-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lambl-1, LGMN, PLVAP, RIN3, ROCK2, SOX7, SOX18, STAB1, STAB2, STUB1, TFEC, THSD1, TNFAIP8, and XLKD1 (LYVE1). Conversely, the step of increasing the number of activated endothelial progenitor cells comprises increasing in the endothelial progenitor cells in the blood of the subject the expression of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYBSB, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lambl-1, LGMN, MMP3, Nos2, PAIL PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYBSB, DLL4, DUSP5, ELK3, ERG1 (KCNH2), ETS1, ETS2, EXOC3L, FGD5, GRRP1, HO-1 (HMOX1), HO-2 (HMOX2), LAMA4, Lambl-1, LGMN, PLVAP, RIN3, ROCK2, SOX7, SOX18, STAB1, STAB2, STUB1, TFEC, THSD1, TNFAIP8, and XLKD1 (LYVE1).

The generation of EPC and EPC-derived endothelial cells is disclosed in the invention through culture of EPC or EPC-derived endothelial cells in conditions which resemble the tumor microenvironment. One such condition is exposure to ionic concentrations which resemble the tumor microenvironment. It is known that tumours contain areas of cellular necrosis, which are associated with poor survival in a variety of cancers. A study showed that necrosis releases intracellular potassium ions into the extracellular fluid of mouse and human tumours, causing profound suppression of T cell effector function. Elevation of the extracellular potassium concentration ([K+]e) impairs T cell receptor (TCR)-driven Akt-mTOR phosphorylation and effector programmes. Potassium-mediated suppression of Akt-mTOR signaling and T cell function is dependent upon the activity of the serine/threonine phosphatase PP2A. Although the suppressive effect mediated by elevated [K+]e is independent of changes in plasma membrane potential (Vm), it requires an increase in intracellular potassium ([K+]i). Accordingly, augmenting potassium efflux in tumour-specific T cells by overexpressing the potassium channel Kv1.3 lowers [K+]i and improves effector functions in vitro and in vivo and enhances tumour clearance and survival in melanoma-bearing mice. In one embodiment the invention teaches the use of culture conditions similar to those associated with necrotic tissue as a means of modifying EPC and EPC-derived endothelial cells to render the cells similar to tumor endothelial cells. In other embodiments of the invention EPC or EPC-derived endothelial cells are cultured under conditions of free adenosine similar to those found in tumor cells. Numerous publications report concentrations found in tumors and several are incorporated by reference. In one embodiment of the invention EPC or endothelial cells derived thereof are cultured with enzymes known to induce production of adenosine locally in a manner similar to that found in the tumor microenvironment. Enzymes, or ectoenzymes useful for the practice of the invention include CD39, and CD73, which are described in the associated references and incorporated herein.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

1. A method of treating cancer comprising the steps of: a) obtaining endothelial progenitor cells; b) culturing the endothelial progenitor cells under conditions resembling the tumor microenvironment; and c) administering products of the cultured endothelial progenitor cells in a manner to stimulate an immune response capable of cross-reacting with tumor associated endothelial cells.

2. The method of claim 1, wherein the endothelial progenitor cell is derived from placental tissue.

3. The method of claim 1, wherein the endothelial progenitor cell is HLA mismatched to the cancer patient in need of treatment.

4. The method of claim 1, wherein the endothelial progenitor cell is treated with interferon gamma at concentrations and duration sufficient to increase expression of HLA I and HLA II.

5. The method of claim 1, wherein the endothelial progenitor cells or progeny thereof are rendered metabolically similar to tumor endothelial cells by culture under hypoxic conditions.

6. The method of claim 1, wherein the endothelial progenitor cells or products thereof are generated to resemble tumor endothelial cells by culture in acidic conditions.

7. The method of claim 6, wherein the acidic conditions comprise a pH of less than about 7.

8. The method of claim 6, wherein the acidic conditions comprise a pH of about 5 to about 7.

9. The method of claim 6, wherein the acidic conditions comprise a pH of about 5.5 to about 6.5.

10. The method of claim 6, wherein the acidic conditions comprise a pH of about 6.

11. The method of claim 6, wherein the cells are cultured under acidic conditions for a timepoint sufficient to induce upregulation of VEGF-R2.

12. The method of claim 6, wherein the cells are cultured under acidic conditions for a timepoint sufficient to induce upregulation of TEM-1.

13. The method of claim 6, wherein the cells are cultured under acidic conditions for a timepoint sufficient to induce upregulation of CD-105.

14. The method of claim 6, wherein the cells are cultured under acidic conditions for a timepoint sufficient to induce upregulation of CD-73.

15. The method of claim 6, wherein the cells are cultured under acidic conditions for a timepoint sufficient to induce upregulation of CD-39.

16. The method of claim 6, wherein the cells are cultured under acidic conditions for a timepoint sufficient to induce upregulation of HLA-G.

17. The method of claim 6, wherein the cells are cultured under acidic conditions for a timepoint sufficient to induce upregulation of FAS-L.

18. The method of claim 1, wherein the endothelial progenitor cells or products thereof are made to resemble tumor endothelium by culture in a media depleted of amino acids in order to activate GCN2 kinase.

19. The method of claim 1, wherein the endothelial progenitor cells or products thereof are made to resemble tumor endothelium by culture in a media containing adenosine.
